(19) United States
(12) Patent Application Publication
(10) Pub. No.: US 2025/0345499 A1
(43) Pub. Date: Nov. 13, 2025

(54) PATIENT BODY TEMPERATURE CONTROL DURING GAS EXCHANGE TREATMENT

(71) Applicant: Maquet Critical Care AB, Solna (SE)

(72) Inventors: Åke LARSSON, Järfälla (SE); Mario LONCAR, Ekerö (SE); Michael BROOMÈ, Solna (SE)

(21) Appl. No.: 18/872,039

(22) PCT Filed: Jun. 5, 2023
(86) PCT No.: PCT/SE2023/050565

§ 371 Date: Dec. 5, 2024

(30) Foreign Application Priority Data
Jun. 10, 2022 (SE) 2250701-6

(51) Int. Cl. A61M 1/16; A61M 16/00

(52) U.S. Cl. CPC A61M 1/1698; A61M 16/0003; A61M 2205/3372; A61M 2230/20; A61M 2230/43; A61M 2230/50

(57) ABSTRACT

A method is for patient body temperature control during gas exchange treatment of a patient, such as extracorporeal membrane oxygenator [ECMO] treatment provided by an ECMO device and/or respiratory treatment provided by a mechanical ventilator. The method comprises the steps of determining a gas exchange being at least one of a carbon dioxide [CO2] exchange and an oxygen [O2] exchange between an oxygen-containing gas and blood of the patient; inducing a change in temperature of the patient; detecting a change in the gas exchange following the change in the temperature of the patient, and automatically controlling the temperature of the patient based on the detected change in gas exchange, and/or presenting a recommendation for manual adjustment of the temperature of the patient to a user, based on the detected change in gas exchange.

### TECHNICAL FIELD

The present disclosure relates to the field of intensive care and, in particular, to a system, method and computer program for patient body temperature control during extracorporeal membrane oxygenation (ECMO) treatment and/or mechanical ventilation.

### BACKGROUND

Mechanical ventilators and medical devices for oxygenation and extracorporeal removal of CO2 from human blood are well known examples of intensive care equipment that are used to provide ventilatory and sometimes circulatory support to patients with reduced lung function.

Mechanical ventilators are used to provide respiratory treatment to patients through the supply of oxygen-containing breathing gas to the patient's lungs, allowing CO2 to be removed from, and oxygen to be added to, the circulatory system of a patient through gas exchange within the lungs.

Historically, medical devices for extracorporeal removal of CO2 from human blood, often referred to as extracorporeal membrane oxygenation (ECMO) devices, have primarily been used to provide ventilatory and circulatory support to patients having reduced lung and/or heart function in situations where conventional and less invasive treatments, such as mechanical ventilation, have been insufficient. Lately, however, combined treatment by ECMO devices and mechanical ventilators have gained more and more interest from clinicians also in the treatment of patients suffering from less severe lung conditions.

In an ECMO device, carbon dioxide rich blood is withdrawn from the patient and provided to an oxygenator that serves as an artificial lung by removing CO2 and adding oxygen to the blood before the oxygen-enriched blood is returned to the circulatory system of the patient. The removal of CO2 and the addition of oxygen is achieved by sweeping an oxygen-containing sweep gas flow through the oxygenator, allowing gas exchange between the blood and the sweep gas to take place over the oxygenator membrane.

During ECMO treatment, the blood in the extracorporeal circuit is heated to compensate for the energy losses from the extracorporeal circulation of blood. The external heating actually overrides the patient's own temperature control if not handled carefully by the clinician.

The normal practice is to manually adjust the heating of the blood returning to the patient in order to keep the patient at a normal body temperature, i.e. about 37 degree Celsius.

In a patient with artificial external heating it is difficult to detect and discern when the patient has fever because the body temp is controlled. When a patient is febrile, the body temperature set point of the patient is shifted up and the physiology responds with an increase in metabolic rate in order to increase the actual body temperature. This is sometimes seen as shivering and muscle activity. The perception of being cold (as everybody has experienced at the onset of fever) is also associated with a significant level of discomfort.

For a patient with no gas exchange margins even during rest, any increase in metabolic demand from stress, anxiety or activity (like shivering) can lead to increasing demands on the cardiovascular and pulmonary system as well as discomfort/panic in a vicious circle since they have no possibility to compensate the increased metabolic demand.

In a similar way, when a patient stops being febrile, the body temperature set point of the patient is shifted down and the physiology responds with a decrease in metabolic rate in order to decrease the actual body temperature. This is sometimes associated with sweating. The perception of being too warm is also associated with a significant level of discomfort.

### SUMMARY

It is an object of the present disclosure to present a method, a computer program and a system for solving or mitigating one or more of the above mentioned problems associated with the prior art.

It is a particular object of the present disclosure to present a method, a computer program and a system for improved patient body temperature control during gas exchange treatments, such as extracorporeal membrane oxygenation (ECMO) treatment and/or mechanical ventilation.

These and other objects, which will become apparent in view of the detailed description following hereinafter, are achieved in accordance with the a system, method and computer program as defined by the appended claims.

According to a first aspect of the present disclosure there is provide a method for patient body temperature control during gas exchange treatment of a patient, such as ECMO treatment provided by an ECMO device and/or respiratory treatment provided by a mechanical ventilator. The method comprises the steps of:
determining a gas exchange being at least one of a carbon dioxide (CO2) exchange and an oxygen (O2) exchange between an oxygen-containing gas and blood of the patient;
inducing a change in temperature of the patient;
detecting a change in the gas exchange following the change in the temperature of the patient, and
automatically controlling the temperature of the patient based on the detected change in gas exchange, and/or
presenting a recommendation for manual control of the temperature of the patient to a user, based on the detected change in gas exchange.

Thus, the present disclosure suggests a manoeuvre in which a change in the temperature of the patient is introduced in order to evaluate whether a current temperature of the patient is an optimal temperature, based on a change in gas exchange caused by the change in patient temperature. If, for example, the gas exchange is reduced in response to the change in patient temperature, then the new temperature is likely to be a more optimal temperature and should be maintained, or the temperature should be further adjusted in the direction of the change in temperature. The temperature may then be controlled accordingly, and/or a recommendation for manual patient temperature control may be presented to a clinician.

According to some embodiments, the method comprises the steps of:
detecting a reduction in gas exchange following a change in temperature of the patient from an original patient temperature to a new patient temperature, and
in response to detecting the reduction in gas exchange, automatically controlling the temperature of the patient such that it is maintained at the new temperature or further adjusted in the direction of the induced change in temperature, and/or
in response to detecting the reduction in gas exchange, presenting a recommendation to manually adjust the temperature of the patient in the direction of the induced change in temperature.

According to some embodiments, the method comprises the steps of:
detecting an increase in gas exchange following a change in temperature of the patient from an original patient temperature to a new patient temperature, and
in response to detecting the increase in gas exchange, automatically controlling the temperature of the patient such that it is maintained at the original temperature or further adjusted in the opposite direction of the induced change in temperature, and/or
in response to detecting the increase in gas exchange, presenting a recommendation to manually adjust the temperature of the patient in an opposite direction of the induced change in temperature.

According to some embodiments, the method comprises the steps of:
obtaining a body temperature of the patient, and
automatically controlling the temperature of the patient based on both the detected change in gas exchange and the body temperature of the patient, and/or
presenting the recommendation for manual control of the temperature of the patient to a user based on both the detected change in gas exchange and the body temperature of the patient.

According to some embodiments, the gas exchange treatment includes ECMO treatment provided by an ECMO device and the step of inducing a change in the temperature of the patient and/or the step of automatically adjusting the temperature of the patient involves adjustment of a temperature of an extracorporeal bloodstream that is recirculated back to the patient after gas exchange between the bloodstream and a sweep gas flow over an oxygenator.

According to another aspect of the present disclosure there is provided a computer program for patient body temperature control during gas exchange treatment of a patient. The computer program comprises computer-readable instructions which, when executed by a control computer, causes the steps of the above described method to be performed.

According to another aspect of the present disclosure there is provided a computer program product comprising a data storage medium, such as a non-transitory memory hardware device, storing the above mentioned computer program for patient body temperature control during gas exchange treatment of a patient.

According to yet another aspect of the present disclosure there is provide a system for patient body temperature control during gas exchange treatment of a patient. The system comprises at least one control computer configured to:
determine a gas exchange being at least one of a CO2 exchange and an O2 exchange between an oxygen-containing gas and blood of the patient;
induce a change in temperature of the patient;
detect a change in the gas exchange following the change in the temperature of the patient, and
automatically control the temperature of the patient based on the detected change in gas exchange, and/or
present a recommendation for manual control of the temperature of the patient to a user, based on the detected change in gas exchange.

According to some embodiments, the control computer is configured to:
detect a reduction in gas exchange following a change in temperature of the patient from an original patient temperature to a new patient temperature, and
in response to detecting the reduction in gas exchange, automatically control the temperature of the patient such that it is maintained at the new temperature or further adjusted in the direction of the induced change in temperature, and/or
in response to detecting the reduction in gas exchange, present a recommendation to manually adjust the temperature of the patient in the direction of the induced change in temperature.

According to some embodiments, the control computer is configured to:
detect an increase in gas exchange following a change in temperature of the patient from an original patient temperature to a new patient temperature, and
in response to detecting the increase in gas exchange, automatically controlling the temperature of the patient such that it is maintained at the original temperature or further adjusted in the opposite direction of the induced change in temperature, and/or
in response to detecting the increase in gas exchange, presenting a recommendation to manually adjust the temperature of the patient in an opposite direction of the induced change in temperature.

According to some embodiments, the control computer is configured to:
obtain a body temperature of the patient, and
automatically control the temperature of the patient based on both the detected change in gas exchange and the body temperature of the patient, and/or
present the recommendation for manual control of the temperature of the patient to a user based on both the detected change in gas exchange and the body temperature of the patient.

According to some embodiments, the system comprises an ECMO device and the control computer is configured to induce the change in temperature of the patient and/or to automatically adjust the temperature of the patient by causing adjustment of a temperature of an extracorporeal bloodstream that is recirculated back to the patient after gas exchange between the bloodstream and a sweep gas flow over an oxygenator.

More advantageous features of the method, computer program and system of the present disclosure will be described in the detailed description following hereinafter.

### BRIEF DESCRIPTION OF THE DRAWINGS

The present invention will become more fully understood from the detailed description provided hereinafter and the accompanying drawings, which are given by way of non-limiting illustration only. In the different drawings, same reference numerals correspond to the same element.

FIG. 1 illustrates an exemplary non-limiting embodiment of an ECMO-vent system for extracorporeal removal of CO2 from the blood of a patient undergoing mechanical ventilation.

FIG. 2 illustrates an exemplary non-limiting embodiment of an ECMO device of the ECMO-vent system in FIG. 1.

FIG. 3 is a flowchart illustrating an exemplary non-limiting embodiment of a method for gas exchange determination.

FIG. 4 illustrates an exemplary non-limiting embodiment of a mechanical ventilation system.

### DETAILED DESCRIPTION

The present disclosure relates to the field of extracorporeal blood gas exchange by use of an oxygenator for extracorporeal removal of carbon dioxide (CO2) from the blood of a patient. In particular, the disclosure relates to a method, a computer program and a system for improved control of CO2 removal by the oxygenator through addition of CO2 to a sweep gas flow through the oxygenator.

The invention will hereinafter be described in the context of a combined system for extracorporeal blood gas exchange via an extracorporeal membrane oxygenator (ECMO) during lung protective ventilation of the patient using a mechanical ventilator. Such a combined system comprising both an ECMO device and a mechanical ventilator will herein be referred to as an ECMO-vent system. However, it should be appreciated that the principles of the present disclosure are equally applicable to a standalone ECMO device and a standalone mechanical ventilator.

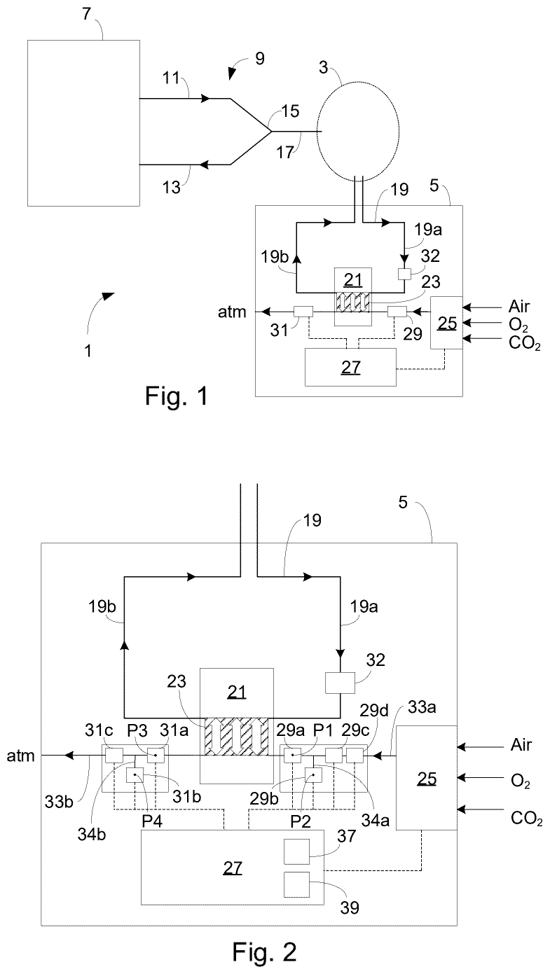

FIG. 1 illustrates a system 1 for combined mechanical ventilation of the lungs of a patient 3 and extracorporeal removal of CO2 from the blood of the patient 3. The system 1 will hereinafter referred to as an ECMO-vent system. ECMO (extracorporeal membrane oxygenation) is one of several terms used for extracorporeal blood gas exchange where blood is pumped outside the body of a treated patient to a device, sometimes referred to as a heart-lung machine, which removes CO2 and sends oxygen-enriched blood back to the patient. Other terms that are frequently used in the art for the same or similar treatments are ECLA (extracorporeal lung assist), ECCO2R (extracorporeal CO2 removal), ECLS (extracorporeal life support) and ECGE (extracorporeal membrane gas-exchange), all of which are encompassed by the term ECMO as used herein.

The ECMO-vent system 1 comprises a device 5, hereinafter referred to as an ECMO device, for extracorporeal removal of CO2 from the blood of the patient 3, and a mechanical ventilator 7 for mechanically ventilating the patient 3 through the supply of breathing gas to the lungs of the patient.

The ventilator 7 comprises or is connected to a source of pressurised breathing gas (not shown), which breathing gas is supplied to the patient 3 via a patient circuit 9. In this example, the patient circuit 9 comprises an inspiratory line 11 for conveying a flow of breathing gas to the patient 3, and an expiratory line 13 for conveying a flow of exhalation gas exhaled by the patient away from the patient. The inspiratory line 11 and the expiratory line 13 are connected to each other via a so called Y-piece 15 which, in turn, is connected to the patient 3 via a common line 17.

The ECMO device 5 is configured to provide ECMO treatment to the patient 3 by generating an extracorporeal flow of blood from the patient 3, oxygenating the blood through extracorporeal blood gas exchange in which CO2 is removed from, and oxygen (O2) added to, the extracorporeal blood flow, and returning the oxygen-enriched blood to the patient 3.

To generate the flow of blood to and from the patient 3, the ECMO device 5 may comprise a blood flow generator (not shown), typically in form of one or several roller, turbine and/or centrifugal pumps. The blood flow generator generates a flow of blood through a tubing system forming a blood flow channel 19 of the ECMO device 5, where parts of the channel may be heated and/or cooled to maintain a desired temperature of the blood when returned to the patient 3.

The blood gas exchange, including blood oxygenation and CO2 removal, takes place in a membrane oxygenator 21 of the ECMO device 5, in which an oxygen-containing sweep gas flow interacts with the blood in the blood flow channel 19 via a membrane 23 of the oxygenator 21. The membrane 23 acts as a gas-liquid barrier enabling transfer of CO2 and O2 content between the bloodstream flowing through the oxygenator 21 on a liquid-side of the membrane 23 and the sweep gas flow flowing through the oxygenator 21 on a gas-side of the membrane 23.

The sweep gas flow is generated by a sweep gas generator 25 connected to one or more sweep gas sources, typically including one or both of an oxygen source and a source of compressed air. According to the principles of the present disclosure, the sweep gas generator 25 is further connected to a CO2 source in order to control the degree of CO2 removal over the oxygenator 21 through addition of CO2 to the sweep gas flow. The sweep gas generator 23 is configured to deliver a controllable sweep gas composition to the oxygenator 21 at a controllable sweep gas flow rate.

The composition and, optionally, the flow rate of the sweep gas generated by the sweep gas generator 23 may be automatically controlled by a controller or control computer 27 of the ECMO device 5 based on set target values and sensor data obtained by various sensors 29, 31 of the ECMO device 5. In particular, the control computer 27 of the ECMO device 5 may be configured to automatically control an addition of CO2 to a sweep gas flow comprising any or both of oxygen and air, based on a set target for a measure of CO2 removal by the oxygenator 21.

Hereinafter, the sweep gas flow upstream of the oxygenator 21 (i.e., before the oxygenator from the sweep gas' point of view) will be referred to as an input sweep gas flow or a pre-oxygenator sweep gas flow, and the sweep gas flow downstream of the oxygenator 21 (i.e., after the oxygenator from the sweep gas' point of view) will be referred to as an output sweep gas flow or a post-oxygenator sweep gas flow. The input sweep gas flow flows from the sweep gas generator 25 to the oxygenator 21 via a sweep gas inlet line 33a of the ECMO device 5, and the output sweep gas flow flows from the oxygenator 21 to atmosphere or an evacuation or recirculation system via a sweep gas outlet line 33b. In most configurations, ECMO systems are open systems, meaning that the post oxygenator sweep gas flow is allowed to escape into the ambient. In some cases, especially when anesthetic agents are added to the sweep gas flow, a closed or semi closed (sweep) gas control system can be envisioned, similar to gas control systems often used in anesthesia machines.

Likewise, the bloodstream upstream of the oxygenator 21 (i.e., before the oxygenator from the bloodstream's point of view) may hereinafter be referred to as an input bloodstream or pre-oxygenator bloodstream, and the bloodstream downstream of the oxygenator 21 (i.e., after the oxygenator from the bloodstream's point of view) may be referred to as an output bloodstream or post-oxygenator bloodstream. The input bloodstream flows from the patient 3 to the oxygenator 21 via a bloodstream inlet line 19a of the ECMO device 5, and the output bloodstream flows from the oxygenator 21 and back to the patient 3 via a bloodstream outlet line 19b of the ECMO device 5.

With reference now made to FIG. 2, the sensors 29, 31 of the ECMO device 5 may comprise:
a pre-oxygenator flow rate sensor 29a for measuring a flow rate of the input sweep gas flow, Vᵢₙ. The pre-oxygenator flow rate sensor 29a is a mainstream flow sensor, meaning that it is configured to measure the flow rate of the sweep gas flowing in the sweep gas inlet line 33a. The pre-oxygenator flow measurements obtained by the pre-oxygenator flow rate sensor 29a take place at a pre-oxygenator point of flow measurement denoted P1 in the sweep gas inlet line 33a.
a pre-oxygenator gas analyser 29b for measuring a fraction of at least CO2 in the input sweep gas flow, FCO2ᵢₙ. The pre-oxygenator gas analyser 29b may also be configured to measure a fraction of one or more additional gases selected from the group consisting of oxygen (O2), nitrogen gas (N2), and anaesthetic agents. In this exemplary embodiment, the pre-oxygenator gas analyser 29b is a so called sidestream gas analyser that is configured to withdraw sweep gas samples from the sweep gas inlet line 33a, and measure the fraction of CO2 and, optionally, the fraction of the at least one additional gas in the sweep gas samples at a pre-oxygenator point of CO2 measurement denoted P2. The pre-oxygenator point of CO2 measurement P2 is separated in distance from the point of pre-oxygenator sweep gas flow rate measurements, P1, at least by the length of a pre-oxygenator sample line 34a. In other embodiments, the pre-oxygenator gas analyser 29b may be a so called mainstream gas analyser that is configured to measure the fraction of CO2 and, optionally, the fraction of the at least one additional gas within the sweep gas inlet line 33a. In some embodiments, the pre-oxygenator gas analyser comprises at least a CO2 sensor and an O2 sensor for measuring a fraction of CO2 and O2, respectively, in the sweep gas samples. In some embodiments, the CO2 sensor is a non-dispersive infrared (NDIR) CO2 sensor. In some embodiments, the O2 sensor is a paramagnetic or electrochemical O2 sensor.
a pre-oxygenator temperature sensor 29c for measuring a temperature of the input sweep gas, T_{in,gas}.
a pressure sensor 29d for measuring a sweep gas circuit pressure, P_{gas}, substantially corresponding to the sweep gas pressure in the gas inlet line 33a.
a post-oxygenator flow rate sensor 31a for measuring a flow rate of the output sweep gas flow, Vₒᵤₜ. The post-oxygenator flow rate sensor 31a is a mainstream flow sensor, meaning that it is configured to measure the flow rate of the sweep gas flowing in the sweep gas outlet line 33b. The post-oxygenator flow measurements obtained by the post-oxygenator flow rate sensor 31a take place at a post-oxygenator point of flow measurement denoted P3 in the sweep gas outlet line 33b.
a post-oxygenator gas analyser 31b for measuring a fraction of at least CO2 in the output sweep gas flow, FCO2ₒᵤₜ. The post-oxygenator gas analyser 31b may also be configured to measure a fraction of one or more additional gases selected from the group consisting of oxygen (O2), nitrogen gas (N2), and anaesthetic agents. In this exemplary embodiment, the post-oxygenator gas analyser 31b is a so called sidestream gas analyser that is configured to withdraw sweep gas samples from the sweep gas outlet line 33b, and measure the fraction of CO2 and, optionally, the fraction of the at least one additional gas in the sweep gas samples at a post-oxygenator point of CO2 measurement denoted P4. The post-oxygenator point of CO2 measurement P4 is separated in distance from the point of post-oxygenator sweep gas flow rate measurements, P3, at least by the length of a post-oxygenator sample line 34b. In other embodiments, the post-oxygenator gas analyser 31b may be a so called mainstream gas analyser that is configured to measure the fraction of CO2 and, optionally, the fraction of the at least one additional gas within the sweep gas outlet line 33b. In some embodiments, the post-oxygenator gas analyser comprises at least a CO2 sensor and an O2 sensor for measuring a fraction of CO2 and O2, respectively, in the sweep gas samples. In some embodiments, the CO2 sensor is an IR spectrometer for IR spectroscopy, such as IR absorption spectroscopy. In some embodiments, the O2 sensor is a paramagnetic O2 sensor.
a post-oxygenator temperature sensor 31c for measuring a temperature of the output sweep gas.

In some embodiments, the ECMO device 5 may further comprise or be connected to a pre-oxygenator blood gas analyser 32 for measuring a partial pressure of at least CO2 in the input bloodstream, PCO2ᵢₙ. The pre-oxygenator blood gas analyser 32 may also be configured to measure a partial pressure of O2 in the input bloodstream, PO2ᵢₙ. The pre-oxygenator blood gas analyser 32 may also be configured to measure a haemoglobin content of the input bloodstream, Hbᵢₙ. In some embodiments, the blood gas analyser 32 is not incorporated into the ECMO device 5 but arranged to form part of another medical device that is connected to the ECMO device 5 in order for the ECMO device 5 to receive measurements obtained by the blood gas analyser. For example, the blood gas analyser may form part of a stand-alone blood gas analyser unit, often referred to as a BGA, commonly used for intermittent blood gas analysis during ECMO treatments.

In accordance with the principles of the present disclosure, the ECMO device 5 and/or the mechanical ventilator 7 may include functionality for patient body temperature control during gas exchange treatment of a patient 3.

In some embodiments, the control computer 27 is configured to:
determine a gas exchange being at least one of a CO2 exchange and an O2 exchange between an oxygen-containing gas and blood of the patient 3;
induce a change in temperature of the patient 3 to assess any change in gas exchange occurring in response to the change in temperature;
detect a change in the gas exchange following the change in the temperature of the patient 3, and
automatically control the temperature of the patient 3 based on the detected change in gas exchange, and/or
presenting a recommendation for manual control of the temperature of the patient 3 to a user, such as a clinician or an operator of the ECMO device 5 and/or the mechanical ventilator 7, based on the detected change in gas exchange.

Thus, the present disclosure suggests a manoeuvre in which a change in the temperature of the patient is introduced in order to evaluate whether a current temperature of the patient is an optimal temperature, based on a change in gas exchange caused by the change in patient temperature. If, for example, the gas exchange (e.g., a total gas exchange of CO2 elimination and O2 uptake, or the O2 uptake of the patient 3) is reduced in response to the change in patient temperature, then the new temperature (after the change) is likely to be a more optimal temperature and should be maintained, or the temperature should be further adjusted in the direction of the change in temperature. The control computer 27 may then control the temperature accordingly, and/or present a recommendation for manual patient temperature control to a clinician, e.g. on a monitor of the system.

The gas exchange is hence typically one of an O2 uptake by the patient 3 and a total gas exchange of CO2 elimination and O2 uptake by the patient.

The metabolic rate as a function of body temperature has a local minimum at or near the patient's optimal body temperature, which optimal body temperature may depend on the physiological state of the patient. A healthy human typically has an optimal body temperature in the range of 36-38 degrees Celsius. More specifically, depending, e.g., on age, time of day, individual variations and the way the temperature is measured, the optimal body temperature of a healthy human is typically in the range of 36,1-37,8 degrees Celsius. On both sides of the local minimum at or near the optimal body temperature, in a range which may be referred to as a normal body temperature range, the body reacts to a change in temperature by increasing the metabolic rate and increasing the O2 uptake, This means that, within the normal body temperature range, if the patient has a body temperature that is higher than the optimal body temperature, the body increases the metabolic rate in response to a further increase in body temperature and decreases the metabolic rate in response to a decrease in body temperature. Likewise, if, within the normal body temperature range, the patient has a body temperature that is lower than the optimal body temperature, the body increases the metabolic rate in response to a further decrease in body temperature and decreases the metabolic rate in response to an increase in body temperature, Thus, within the normal body temperature range, a reduction in gas exchange (indicating a reduced metabolic rate) in response to a change in temperature indicates that the change in temperature has brought the body temperature of the patient closer to the patient's optimal body temperature and hence that the new temperature should be maintained or further changed in the same direction. In this context, the normal body temperature range is typically around 35-41 degrees Celsius.

On the other hand, when the body temperature is outside the normal body temperature range, for example if the patient suffers from hypothermia or hyperthermia, the body's ability to regulate temperature and oxygen uptake is typically impaired. Within these non-normal hypothermic and hyperthermic body temperature ranges, the body's response to a change in temperature is somewhat different. For example, in the hypothermic temperature range (i.e., the temperature range below the normal body temperature range), a further decrease in temperature results in a decrease in metabolic rate, whereas an increase in temperature results in an increase in metabolic rate.

Considering the above, it may be important to know the body temperature of the patient in order to know how to interpret the gas exchange response to the change in temperature.

Consequently, in some embodiments, the control computer 27 may be configured to:
obtain a body temperature of the patient 3;
induce a change in temperature of the patient 3 to assess any change in gas exchange occurring in response to the change in temperature;
detect a change in the gas exchange following the change in the temperature of the patient 3, and
automatically control the temperature of the patient 3 based on both the detected change in gas exchange and the body temperature of the patient 3, and/or
presenting a recommendation for manual control of the temperature of the patient 3 to a user, such as a clinician or an operator of the ECMO device 5 and/or the mechanical ventilator 7, based on both the detected change in gas exchange and the body temperature of the patient.

The body temperature may be the original body temperature of the patient prior to the change in temperature, or it may be the body temperature of the patient after the change in temperature. The body temperature, the change in body temperature and the gas exchange response to the change in temperature may be used by the control computer 27 to identify where along the body temperature-metabolic rate curve the body temperature of the patient is located, and hence whether the body temperature of the patient should be maintained, increased or decreased.

Most often, however, it can be assumed that the body temperature of the patient is within the normal body temperature range, whereby the control computer 27 may be configured to:
detect a reduction in gas exchange following a change in temperature of the patient 3 from an original patient temperature to a new patient temperature, and
in response to detecting the reduction in gas exchange, automatically control the temperature of the patient 3 such that it is maintained at the new temperature or further adjusted in the direction of the induced change in temperature, and/or
in response to detecting the reduction in gas exchange, present a recommendation to manually adjust the temperature of the patient 3 in the direction of the induced change in temperature.

Likewise, the control computer 27 may be configured to:
detect an increase in gas exchange following a change in temperature of the patient 3 from an original patient temperature to a new patient temperature, and
in response to detecting the increase in gas exchange, automatically control the temperature of the patient 3 such that it is maintained at the original temperature or further adjusted in the opposite direction of the induced change in temperature, and/or
in response to detecting the reduction in gas exchange, present a recommendation to manually adjust the temperature of the patient 3 in an opposite direction of the induced change in temperature.

In some embodiments, the control computer 27 may be configured to determine, based on the obtained body temperature of the patient 3, if the body temperature of the patient is within the normal body temperature range, and to respond to the reduction or increase in gas exchange in the above described manner only if the body temperature of the patient is within the normal body temperature range.

In some embodiments, the control computer 27 may be configured to determine, based on the obtained body temperature of the patient 3, that the body temperature of the patient is within a hypothermic body temperature range, and to increase the body temperature until it falls within the normal body temperature range.

Likewise, in some embodiments, the control computer 27 may be configured to determine, based on the obtained body temperature of the patient 3, that the body temperature of the patient is within a hyperthermic body temperature range, and to decrease the body temperature until it falls within the normal body temperature range.

Consequently, the control computer 27 of the present disclosure is configured to detect a gas exchange response to an induced change in temperature, and to change the body temperature of the patient based on the response in order to push the body temperature of the patient in the direction of an optimal body temperature of the patient, corresponding to a local minimum of the metabolic rate as a function of body temperature.

In embodiments where the gas exchange treatment includes ECMO treatment provided by the ECMO device 5, the control computer 27 may be configured to induce the change in the temperature of the patient 3 and/or to automatically adjust the temperature of the patient 3 by adjusting a temperature of an extracorporeal bloodstream that is recirculated back to the patient 3 after gas exchange between the bloodstream and the sweep gas flow over the oxygenator 21.

The temperature change may be any of an increase or a decrease in temperature.

Monitored changes in gas exchange may be used also for other than immediate temperature control. For example, the control computer 27 may be configured to predict future changes in patient temperature based on a current change in gas exchange. If the metabolic demand changes in an otherwise stable patient, the change can be an indication of fever and a forthcoming infection, or other change in clinical status of the patient. Therefore, changes in gas exchange indicative of future changes in body temperature of the patient can be used to foresee changes in the clinical status of the patient, allowing a clinician to take precautionary measures and give the patient proper treatment.

To assist the clinician in this regard, the control computer 27 may be configured to:
monitor a gas exchange being at least one of a CO2 exchange and an O2 exchange between an oxygen-containing gas and blood of the patient 3;
detect a change in the gas exchange,
predict a future change in temperature of the patient based on the detected change in gas exchange, and
present information on the predicted change in patient temperature to a user, and/or
automatically adjust an ongoing treatment of the patient 3 based on the predicted change in patient temperature, and/or.
present a recommendation to the user to perform a manoeuvre involving inducing a change in temperature of the patient and monitoring the response in gas exchange, as described above.

The CO2 exchange and/or the O2 exchange may be determined using any known techniques for determination of CO2 and O2 exchange. There are many known techniques for determining, e.g., CO2 elimination and O2 uptake in patients connected to ECMO devices and/or mechanical ventilators.

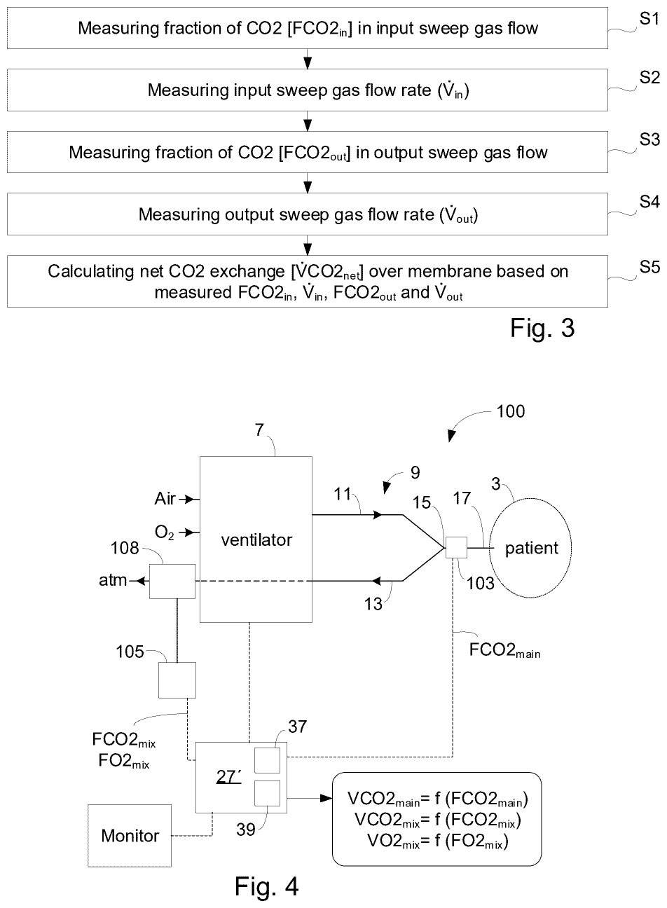

For example, as illustrated by the flowchart in FIG. 3, the CO2 exchange over the oxygenator 21 of the ECMO device 5 may be determined through a method for net CO2 exchange (VCO2ₙₑₜ) calculation, comprising the steps of:
S1) measuring a pre-oxygenator fraction of CO2 (FCO2ᵢₙ) in the sweep gas flow upstream of the oxygenator 21, e.g., by means of the pre-oxygenator gas analyser 29b;
S2) measuring a pre-oxygenator sweep gas flow rate (Vᵢₙ) of the sweep gas flow upstream of the oxygenator 21, e.g., by means of the pre-oxygenator flow rate sensor 29a;
S3) measuring a post-oxygenator fraction of CO2 (FCO2ₒᵤₜ) in the sweep gas flow downstream of the oxygenator 21, e.g., by means of the post-oxygenator gas analyser 31b;
S4) measuring a post-oxygenator sweep gas flow rate (Vₒᵤₜ) of the sweep gas flow downstream of the oxygenator 21, e.g., by means of a post-oxygenator flow rate sensor 31a, and
S5) calculating VCO2ₙₑₜ over the membrane 23 based on measured FCO2ᵢₙ, Vᵢₙ, FCO2ₒᵤₜ and Vₒᵤₜ.

The same principle can be used for measuring O2 exchange over the oxygenator 21 since the pre-and post-oxygenator gas analysers 29b and 31b are configured for both CO2 and O2 measurements. The functionality for determining the gas exchange over the oxygenator 21 may reside in the ECMO device 5 or another device to which the ECMO device 5 is connected.

FIG. 4 illustrates a more detailed view of the mechanical ventilator 7 comprised in a ventilation system 100. The ventilation system 100 comprises a first gas analyser 103 configured for mainstream capnography and arranged to measure a fraction of carbon dioxide (FCO2ₘₐᵢₙ) in expiration gases exhaled by the patient 3.

The first gas analyser 103 is a capnometer arranged for mainstream (i.e., non-diverting) capnography, meaning that it is configured to measure CO2 at the sample site. The first gas analyser 103 is typically positioned at or near the airway of the patient 3. For example, the first gas analyser 103 may be positioned in the common line 17 of the breathing circuit 9, in close proximity of the Y-piece 15. The first gas analyser 103 is configured to measure a fraction of CO2 at least in the expiration gases exhaled by the patient 3. Typically, the first gas analyser 103 is configured to measure a fraction of CO2 also in the breathing gas inhaled by the patient 3.

The system may further comprise at least one second gas analyser 105. The at least one second gas analyser 105 may comprise an O2 sensor for measuring a fraction of O2 in expiration gases exhaled by the patient, which measurement may be used together with flow measurements and/or information on ventilator minute ventilation to determine a measure of O2 uptake in the lungs of the ventilated patient.

In this exemplary embodiment, the at least one second gas analyser 105 is configured to measure both a fraction of CO2 and a fraction of O2 in expiration gases exhaled by the patient 3. The second CO2 measure (the first being obtained by the first gas analyser 103) can be used to determine a second measure of CO2 elimination of the patient, which measure can be compared to a first measure of CO2 elimination determined from the mainstream capnography measurements obtained by the first gas analyser 103 in order to validate or compensate the measure of O2 uptake determined from the O2 measurements obtained by the second gas analyser 105.

The at least one second gas analyser 105 may be a single multigas analyser for measuring both CO2 and O2, or it may be two separate gas analysers for measuring CO2 and O2, respectively. In one example, the at least one second gas analyser 105 is a single gas analyser comprising a non-dispersive infrared (NDIR) CO2 sensor for CO2 measurements and a paramagnetic or electrochemical O2 sensor for O2 measurements. The at least one second gas analyser 105 is different than the first gas analyser 103. As will be discussed in more detail below, the at least one second gas analyser 105 may, for example, be arranged to measure CO2 and O2 in expiration gas that is mixed in a mixing chamber coupled to the expiratory line 13, or be a sidestream gas analyser that is arranged to sample expiration gases from, e.g., the common line 17.

The control computer 27′ may be integrated into the ventilator 7, or it may be a stand-alone computer or a computer of any other device to which the ventilator 7 is connected. The control computer 27′ is coupled to the ventilator 7, the first gas analyser 103 and the at least one second gas analyser 105, and is configured to receive measurements obtained by the first 103 and the at least one second 105 gas analyser, as well as various ventilation-related data including current ventilator settings and sensor data obtained by other sensors (not shown) of the ventilation system 1. Among other things, the ventilation-related data may include data on a set fraction of inspired O2 (FiO2), a set or measured minute ventilation, and flow measurements obtained by various flow sensors of the ventilation system 1, which data may be used by the control computer 27′ together with the CO2 and O2 measurements in the calculation of CO2 elimination and O2 uptake.

The control computer 27′ is configured to determine, based on the CO2 measurements obtained by the first gas analyser 103, a first measure of carbon dioxide elimination of the patient 3. This may be achieved by the control computer 27 by integrating a continuously measured or estimated flow of expiration gas through the mainstream gas analyser 105 and a continuously measured fraction of CO2 in the expiration gas flowing through the mainstream gas analyser 105.

The control computer 27 is further configured to determine, based on the measurements obtained by the at least one second gas analyser 105, a second measure of CO2 elimination of the patient 3, as well as a measure of O2 uptake of the patient 3.

The above examples are merely examples of how CO2 elimination and O2 uptake in the ECMO oxygenator 21 and the lungs of the patient can be determined, and the present disclosure is not limited to any particular way of doing so.

1-12. (canceled)

13. A method for patient body temperature control during gas exchange treatment of a patient, such as extracorporeal membrane oxygenator [ECMO] treatment provided by an ECMO device and/or respiratory treatment provided by a mechanical ventilator, comprising the steps of:
determining a gas exchange being at least one of a carbon dioxide [CO2] exchange and an oxygen [O2] exchange between an oxygen-containing gas and blood of the patient;
inducing a change in temperature of the patient;
detecting a change in the gas exchange following the change in the temperature of the patient, and
automatically controlling the temperature of the patient based on the detected change in gas exchange, and/or
presenting a recommendation for manual control of the temperature of the patient to a user, based on the detected change in gas exchange.

14. The method of claim 13, further comprising the steps of:
detecting a reduction in gas exchange following a change in temperature of the patient (3) from an original patient temperature to a new patient temperature; and
in response to detecting the reduction in gas exchange, automatically controlling the temperature of the patient such that it is maintained at the new temperature or further adjusted in the direction of the induced change in temperature; and/or
in response to detecting the reduction in gas exchange, presenting a recommendation to manually adjust the temperature of the patient in the direction of the induced change in temperature.

15. The method of claim 13, further comprising the steps of:
detecting an increase in gas exchange following a change in temperature of the patient from an original patient temperature to a new patient temperature, and
in response to detecting the increase in gas exchange, automatically controlling the temperature of the patient such that it is maintained at the original temperature or further adjusted in the opposite direction of the induced change in temperature, and/or
in response to detecting the increase in gas exchange, presenting a recommendation to manually adjust the temperature of the patient in an opposite direction of the induced change in temperature.

16. The method of claim 13, further comprising the steps of:
obtaining a body temperature of the patient; and
automatically controlling the temperature of the patient based on both the detected change in gas exchange and the body temperature of the patient; and/or
presenting the recommendation for manual control of the temperature of the patient to a user,
wherein the recommendation is based on both the detected change in gas exchange and the body temperature of the patient.

17. The method of claim 13, wherein the gas exchange treatment includes extracorporeal membrane oxygenator [ECMO] treatment provided by an ECMO device and wherein the step of inducing a change in the temperature of the patient and/or the step of automatically adjusting the temperature of the patient involves adjustment of a temperature of an extracorporeal bloodstream that is recirculated back to the patient after gas exchange between the bloodstream and a sweep gas flow over an oxygenator.

18. A computer program for patient body temperature control during gas exchange treatment of a patient, the computer program comprising computer-readable instructions which, when executed by a control computer, causes the method of claim 13 to be performed.

19. A computer program product comprising a non-transitory memory hardware device storing a computer program for patient body temperature control during gas exchange treatment of a patient, the computer program comprising computer-readable instructions which, when executed by a control computer, causes the method of claim 13 to be performed.

20. A system for patient body temperature control during gas exchange treatment of a patient, comprising:
at least one control computer configured to:
determine a gas exchange being at least one of a carbon dioxide [CO2] exchange and an oxygen [O2] exchange between an oxygen-containing gas and blood of the patient;
induce a change in temperature of the patient;
detect a change in the gas exchange following the change in the temperature of the patient; and
automatically control the temperature of the patient based on the detected change in gas exchange, and/or
present a recommendation for manual control of the temperature of the patient to a user, based on the detected change in gas exchange.

21. The system of claim 20, wherein the control computer is configured to:
detect a reduction in gas exchange following a change in temperature of the patient from an original patient temperature to a new patient temperature, and
in response to detecting the reduction in gas exchange, automatically control the temperature of the patient such that it is maintained at the new temperature or further adjusted in the direction of the induced change in temperature, and/or
in response to detecting the reduction in gas exchange, present a recommendation to manually adjust the temperature of the patient in the direction of the induced change in temperature.

22. The system of claim 20, wherein the control computer is configured to:
detect an increase in gas exchange following a change in temperature of the patient from an original patient temperature to a new patient temperature, and
in response to detecting the increase in gas exchange, automatically controlling the temperature of the patient such that it is maintained at the original temperature or further adjusted in the opposite direction of the induced change in temperature, and/or
in response to detecting the increase in gas exchange, presenting a recommendation to manually adjust the temperature of the patient in an opposite direction of the induced change in temperature.

23. The system of claim 20, wherein the control computer is configured to:
obtain a body temperature of the patient, and
automatically control the temperature of the patient based on both the detected change in gas exchange and the body temperature of the patient, and/or
present the recommendation for manual control of the temperature of the patient to a user based on both the detected change in gas exchange and the body temperature of the patient.

24. The system of claims 20, wherein the system comprises an ECMO device and the control computer is configured to induce the change in temperature of the patient and/or to automatically adjust the temperature of the patient by causing adjustment of a temperature of an extracorporeal bloodstream that is recirculated back to the patient after gas exchange between the bloodstream and a sweep gas flow over an oxygenator.
